(19) United States
(12) Patent Application Publication
(10) Pub. No.: US 2025/0049999 A1
(43) Pub. Date: Feb. 13, 2025

(54) DIALYSIS DEVICE HAVING A COMPACT HYDRAULIC UNIT

(71) Applicant: FRESENIUS MEDICAL CARE DEUTSCHLAND GMBH, Bad Homburg (DE)

(72) Inventors: Winfried BREHM, Hofheim (DE); Matthias FEHLER, Wurzburg (DE); Florian WOHLFEIL, Niederwerrn (DE); Benedict GLASER, Schweinfurt (DE)

(73) Assignee: FRESENIUS MEDICAL CARE DEUTSCHLAND GMBH, Bad Homburg (DE)

(21) Appl. No.: 18/721,671

(22) PCT Filed: Dec. 19, 2022
(86) PCT No.: PCT/EP2022/086695

§ 371 Date: Jun. 19, 2024

(30) Foreign Application Priority Data
Dec. 23, 2021 (DE) 10 2021 134 544.8

(51) Int. Cl. A61M 1/16; B29C 64/30; B29L 23/00; B29L 31/00; B33Y 40/20; B33Y 80/00

(52) U.S. Cl. CPC A61M 1/1601; B29C 64/30; B33Y 40/20; B33Y 80/00; A61M 2207/10; B29K 2995/0015; B29L 2023/007; B29L 2031/7546

(57) ABSTRACT

The present invention relates to a medical device, in particular to a dialysis machine, having a hydraulic unit that has a pipe system that is produced by means of an additive production process and whose intermediate spaces are at least partially filled by a matrix. The invention further relates to an associated method.

The present invention relates to a medical device, in particular to a dialysis machine, having a compact hydraulic unit and to a method of producing such a hydraulic unit.

Hydraulic units for medical devices are known from the prior art in which individual fluidic interfaces are connected to one another by means of loose, flexible tubes.

Such arrangements are, however, complex and prone to error in assembly, produce delays, e.g. in pressure hold tests and thus in operation of a medical device, due to the small compressive strength of the lines used, and are additionally also objectionable under hygienic aspects due to the larger number of open lines used.

Against this background, it is the underlying of the present invention to provide a hydraulic unit of a medical device that alleviates or fully eliminates the disadvantages of the prior art. A compact hydraulic unit that is easy to assembly and that satisfies the highest hygiene standards should in particular be provided.

This object is achieved by the subject matters of the independent claims. Advantageous further developments of the present invention form the subject matter of the dependent claims.

A medical device, in particular a dialysis machine, is accordingly provided that is equipped with a hydraulic unit that has a pipe system that is produced by means of an additive production process and whose intermediate spaces are at least partially filled by a matrix.

The hydraulic unit is preferably tube-free, i.e. pipes are used for the fluidic connection instead of tubes. Unlike tubes, pipes are preferably produced from a stiffer or more rigid material and are therefore characterized by an improved compressive strength. This has a positive effect on the performance of pressure hold tests.

The production by means of an additive production process provides a high degree of design freedom so that a one-piece pipe system can be produced in accordance with almost any desired specifications by which the necessity of the plurality of open lines and corresponding seal points is dispensed with, whereby a hydraulic unit in accordance with the invention can be classified as particularly hygienic.

The necessity of installing a plurality of tubes manually by plugging in is additionally dispensed with by the preferably one-piece pipe system. The assembly is thus greatly simplified and can be automated.

In accordance with a preferred embodiment of the invention, the pipe system is not self-supporting and is supported or mechanically stabilized by the matrix.

This makes is possible to design the pipe system with walls that are as thin as possible and therefore in a material-saving manner without having to dispense with brittle materials in the production of the pipe system. In other words, the pipe system is preferably reduced to its substantial function of providing an interface with the fluidic system of the medical device and the provision of the required mechanical stability takes place by the matrix.

In addition to its function for a mechanical stabilization of the pipe system, the matrix can e.g. have a thermally and/or mechanically insulating and/or damping effect and/or can serve as leak protection and/or to reduce mechanical vibrations.

The pipe system is preferably produced by means of 3D printing, in particular by means of continuous liquid interface production, laser sintering, or another additive process. These production processes offer the advantage of very high design freedom since any desired pipe system can be printed.

Fluidic connections such as pipes are preferably printed in one piece free of seals. The total pipe system of the hydraulic unit is preferably produced or printed in one piece. The compact hydraulic unit can hereby be installed with only a few movements of the hand and leak points that cause potential contaminations are very largely avoided. The hydraulic unit can be connected to the medical device via predetermined coupling connectors.

Provision is made in accordance with a preferred embodiment, that the matrix comprises or consists of a foam and/or a molding compound.

The pipe system can serve as a skeleton or frame that is, for example, arranged in or at a housing and is thereupon foamed over or is overmolded by a molding compound, whereby the matrix is formed that preferably partially or completely fills the intermediate spaces of the pipe system and/or intermediate spaces between the pipe system and the housing. The hardening of the foam or of the molding compound can take place by means of UV light, temperature, or in another manner.

The housing can be at least partially formed in one piece with the pipe system by means of an additive production process. The housing can alternatively be designed separately from the pipe system.

It has proven advantageous in practice for the foam to have closed pores and/or to be a polyurethane foam. It would, however, also be conceivable to use an open-pore foam or to produce the foam from a different material.

Provision is made in accordance with an advantageous embodiment that the pipe system is produced from a material, preferably a plastic, in particular a cyanate ester, satisfying medical demands and preferably suitable for hot disinfection and that the matrix is produced from a different material, preferably a plastic, that does not satisfy the medical demands.

This embodiment has the advantage that only the components of the hydraulic unit that come directly into contact with the fluid circuit of the medical device, in particular the pipe system, are composed of a material satisfying medical demands.

Since materials satisfying medical demands are typically more expensive than other materials, the costs of the hydraulic unit can hereby be reduced since the wall thicknesses of the hydraulic connections can be designed in a reduced form and material can thereby be saved. In accordance with the invention, such a more expensive invention is thus only used directly where it is absolutely necessary, for example at the interface to the fluidics, while the function of the mechanical stabilization of the pipe system is taken over by the less expensive matrix material.

A further aspect of the present invention relates to a method of producing a hydraulic unit of a medical device, in particular of a dialysis machine, comprising the steps:
producing a pipe system by means of an additive production process;
at least partially filling intermediate spaces of the pipe system by means of a matrix.

The pipe system is here preferably arranged in a housing that is filled with the matrix prior to the filling.

The pipe system is preferably not self-supporting and is supported or mechanically stabilized by the matrix. In other words, the pipe system serves as a skeleton or as a matrix that is stabilized by the matrix. The pipe system is preferably designed as tube-free in accordance with the invention; the pipe lines of the pipe system are rather printed directly, for example by 3D printing.

Provision is preferably made that the pipe system is produced by means of 3D printing, in particular by means of continuous liquid interface production, laser sintering, or another additive process.

It has furthermore been found to be of advantage in practice if the pipe system is produced from a material, preferably a plastic, in particular a cyanate ester, satisfying medical demands and preferably suitable for hot disinfection and if the matrix is produced from another material, preferably plastic, that does not satisfy the medical demands.

It is pointed out at this point that the terms “a” and “one” do not necessarily refer to exactly one of the elements, even though this represents a possible embodiment, but can also designate a plurality of elements. The use of the plural equally also includes the presence of the element in question in the singular and, conversely, the singular also includes a plurality of the elements in question.

The present disclosure is furthermore not restricted to the explicitly named features and embodiments; it is rather obvious to the skilled person that said features and embodiments are also covered by the present disclosure in isolation, but also in any desired combination.

Further advantages, feature, and effects of the present invention result from the following description of embodiments of the invention with reference to the Figures. There are shown:

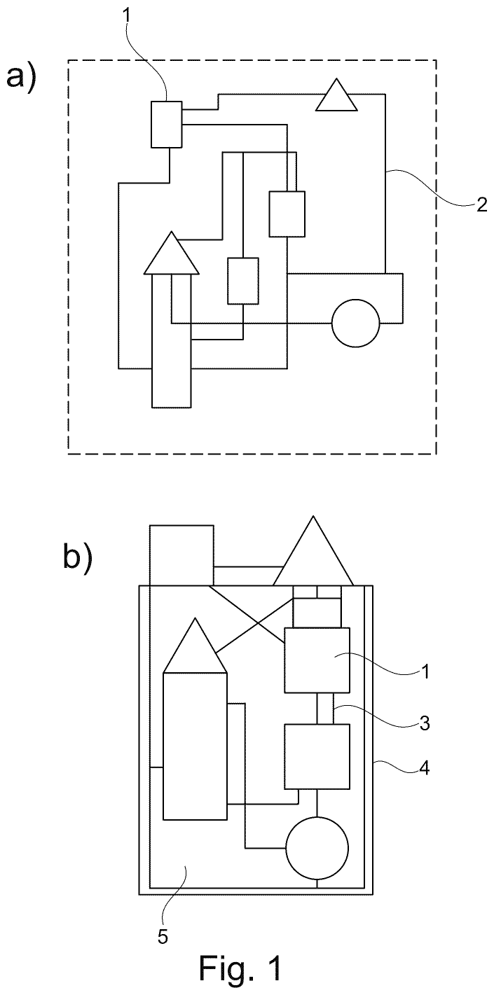

FIG. 1: a schematic comparison of a hydraulic unit in accordance with the prior art (panel a) and a highly integrated hydraulic unit in accordance with the invention (panel b);

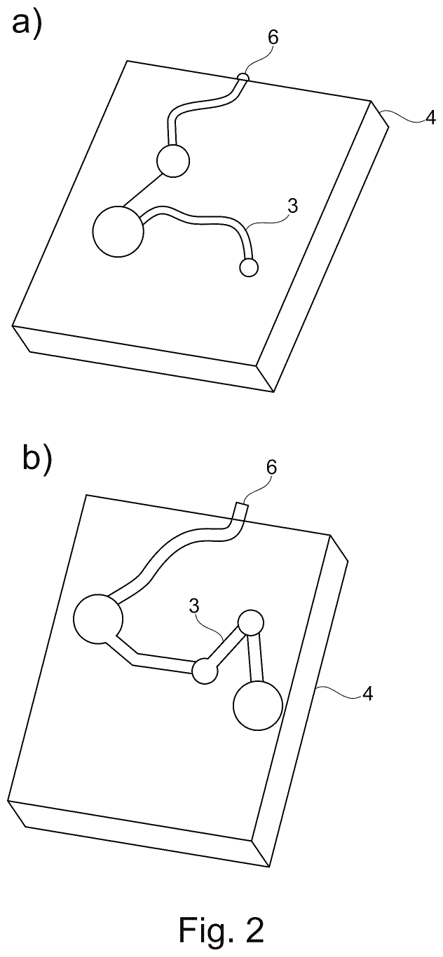

FIG. 2a: pipe system in accordance with the invention that is formed in one piece with a housing;

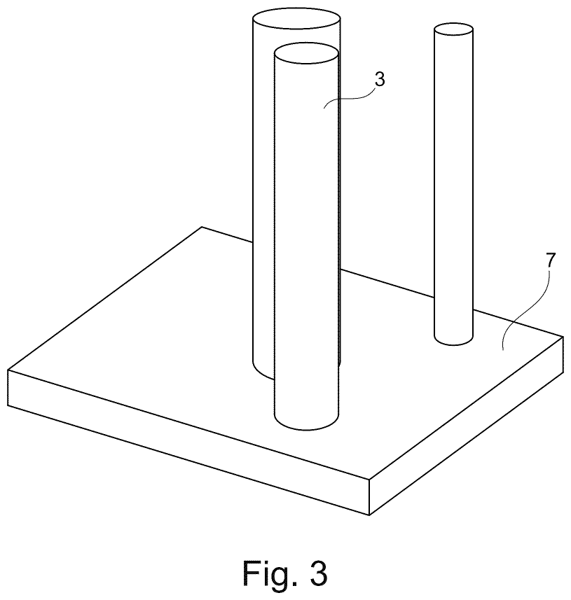

FIG. 3a: pipe system in accordance with the invention that is formed as a skeleton separate from a housing; and

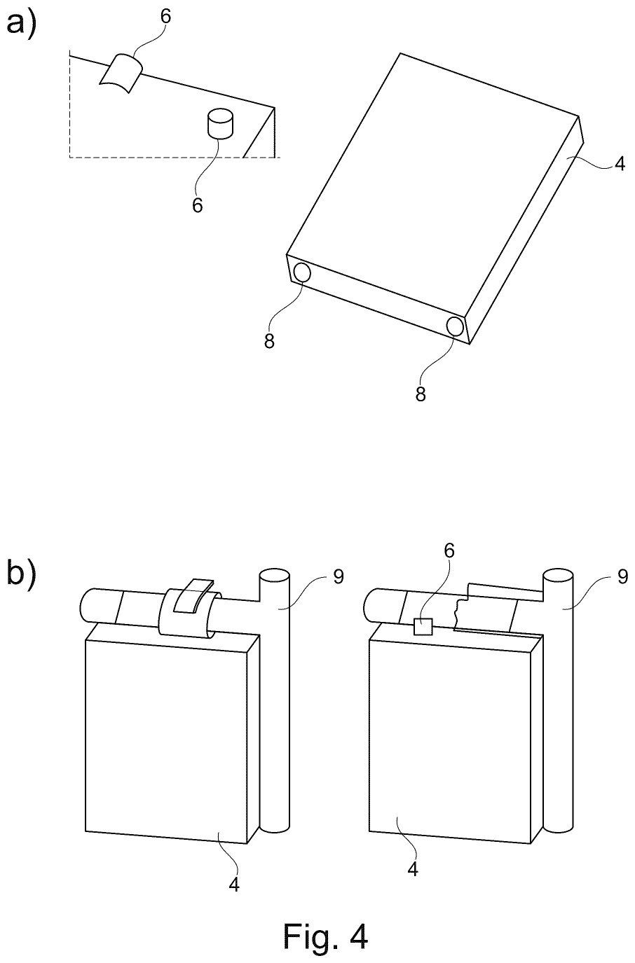

FIG. 4: the pipe system in accordance with the invention of FIG. 2 that is connected to a hydraulic connector of a dialysis machines (matrix is not shown).

As shown in panel a) in FIG. 1, hydraulic connectors 1 that are spaced relatively far from one another in dialysis machines are conventionally connected to one another by means of tubes 2, e.g. of PVC or silicone.

Chambers and other plastic components for conducting fluids are here typically produced in an injection molding process and are sealed by shaped-matched seals (O ring, gasket). The connections between the individual hydraulic components or connector parts are as a rule not optimized with respect to inner fluid volumes, tube lengths, and mechanical stiffness/compressive strength. Such arrangements known from the prior art suffer from the following disadvantages:
Installation may be difficult to automate since the components are installed from different directions. The connections take place by means of silicone tubes that equally have to be plugged on from different directions. The plugging-in is additionally a complex movement process. The plug-in process is therefore performed by hand. The total installation is therefore cost- and time-intensive.
A comparatively large inner volume of the fluidic connections. It follows from this:
Increased energy requirement when heating up; longer heating and cooling times in hot cleaning processes
A larger inner surface causes a larger surface for adhesions such as a biofilm, which is hygienically disadvantageous
A large inner volume means a large distribution volume and thus a longer time period until the temperature and the conductivity in the fluid system of a dialysis machine have been adopted at the start of the treatment or on a change of the parameters in the physiological area
Long cycle times are thus required before, during, and after the treatment for tests and cleaning programs of the fluidic system of the dialysis machine
The use of tubes moreover produces a smaller compressive stiffness of the hydraulic connections. This produces the following disadvantages:
Longer duration of the pressure hold tests
Longer interruption of the treatment (increased time period for cyclic pressure hold test)
Longer preparation time before the treatment (increased time period for initial T1 test) To ensure the hygiene of a dialysis machine, a thermochemical or a purely chemical disinfection is typically carried out on site. That means that the fluid in the hydraulics is heated. It is necessary for the disinfection of a dialysis machine that a fluid temperature above 80° C. is maintained for at least 10 min so that a sufficient disinfection effect is ensured. The demand applies to every branch of the hydraulics.

The connection of the individual components in accordance with the conventional design in FIG. 1a) by open lines has a plurality of problems for the thermal/thermochemical disinfection:
The construction space for hydraulic connections is not separated from the construction space for electromechanical drives, electric sensors, and control electronics, for example of the dialysis machine. The spaces are therefore difficult to separate from one another thermally, if at all.
The electromechanical drives, electric sensors, and the control electronics are exposed to greatly increased environmental temperatures (>65° C.) during the disinfection. Almost all the components of this construction type include semiconductors today and are therefore only specified for operation up to an environmental temperature of approximately 50° C. If this temperature limit is exceeded, the service life is reduced or the failure likelihood of the electronic components is increased.
If a lower operating temperature for the electronic components is provided by sufficient ventilation, the hydraulic connections are simultaneously cooled and the fluid temperature required for disinfection is not reached or is only reached by use of more powerful heatings.

A preferably highly integrated hydraulic unit such as is shown in FIG. 1b) is thus provided in accordance with the invention that alleviates or even fully eliminates the disadvantages of the prior art.

The embodiment of FIG. 1b) is based on the consideration of how components having a fluidic interface can be arranged and fastened in the space in as compact a manner as possible and with connection lengths that are as small as possible. All the components having a fluidic interface have to be arranged as close as possible to one another for this purpose. Hydraulics that follow these specifications are preferably called highly integrated hydraulics.

As shown in FIG. 1b), all the components having a hydraulic interface or all the connectors 1 having connections 3 that are as short as possible and designed as pipes are preferably connected to reduce the inner fluid volume of the hydraulic unit as much as possible. The shorter distances additionally allow a reduction of the connection cross-sections. The fluid volume is thus further reduced. No tubes or at least as few tubes as possible that are as short as possible are preferably used.

As shown in FIG. 1b), the hydraulic unit is of a compact design and is accommodated in a housing 4. The intermediate spaces between the pipes 3 or the pipe system of the hydraulic unit and the housing 4 are filled with a matrix 5 of foam in this embodiment.

Such a highly integrated hydraulic unit provides the following advantages:
The construction space for hydraulic connections is separated from the construction space for electromechanical drives, electric sensors, and control electronics. The construction space for hydraulic connections is additionally optionally insulated (e.g. by foam). The electric components are thus, for example, thermally loaded considerably less during a hot disinfection, which increases the life, reduces the failure rate, and enables the use of less expensive components.
Fewer plug-in tube connections are required and the installation of the components only takes place via predetermined connector points at the outer surfaces of the hydraulic unit or its housing. All the installation points (screw connections) are thus simply reachable, whereby the installation is designed to be able to be automated and inexpensive.
A smaller inner fluid volume and lower compliance enable shorter test times for pressure hold tests. The times for the adjustment/stabilization of the conductivity and the temperature on the initial T1 test of a dialysis machine are likewise shortened.
A smaller inner volume additionally makes possible shorter heating/cooling times and shorter flushing times. The disinfectant consumption and the energy consumption for heating thereby fall and time is furthermore saved.
A smaller inner fluidic surface advantageously means less energy loss in the treatment and disinfection.
More compact hydraulics with less weight and a considerably reduced construction space makes lighter and more compact devices possible and the replacement of the total hydraulic unit is additionally possible in the service case.

FIG. 2 shows in panels a) and b) a pipe system 3 in accordance with the invention that is produced in one piece with a box-like housing 4 by means of 3D printing.

In other words, FIG. 2 shows a 3D printed hydraulic system in panels a) and b) that is designed as a housing/box having inwardly disposed hydraulic connections.

The installation of connectors of a dialysis machine takes place via outwardly disposed connector points 6 of the housing 4. The installation directions are thus thereby clearly defined. Drives, control electronics, etc. of the dialysis machine are also arranged outside the housing 4.

Alternatively, the pipe system 3 can, as shown in FIG. 3, be formed as a skeleton separately from a housing 4.

In this embodiment, the pipe system 3 has at least one base plate 7 that can form a part of the housing 4. For example, a housing 4 could be pushed over the pipe system 3 and either be removed again after the hardening of the matrix material or could be connected to the base plate 7 so that the pipe system is completely surrounded.

As FIG. 4a shows, a housing 4 used as part of the invention has, in addition to the connector points 6 for coupling to a dialysis machine, openings 8 for the introduction of the material forming the matrix (e.g. foam or other molding compound) into the housing 4.

To further insulate the hydraulic connections, to seal them in the event of a defect (leak of the pipe system), and to mechanically stabilize the pipe system, a foam or a molding compound is in other words filled into the space between the hydraulic connections and is then hardened (chemically or thermally).

FIG. 4b shows a hydraulic unit in accordance with the invention in which the hydraulic connectors 9 of a dialysis machine are connected to the housing 4. The construction space for hydraulic connections (within the housing 4 in FIG. 4) is clearly separated from the construction space for the connectors 9 (e.g. associated electromechanical drives, electric sensors, and control electronics; outside the housing 4 in FIG. 4) of the dialysis machine.

The limits of the foamed construction space are set either by a wall that surrounds the hydraulic lines (e.g. the housing 4 or a sheath around the lines) or the total structure of the pipe system is introduced into a mold and the latter is foamed. After the hardening of the foam, the mold can be removed.

The provision of a 3D printed pipe system that can have a housing or can be produced as a skeleton and of a matrix or foam or molding cast surrounding it in particular offers the following advantages in fluid systems where hygiene is critical:
The pipe system produced by 3D printing forms the hydraulic connection
Maximum degrees of design freedom are present on a production in a 3D printing process; a good flushing capability of the hydraulic lines can thus be achieved by the avoidance of undercuts, for example
A configuration of the matrix by cast/foam provides very good thermal insulation (even shorter cycle times, in particular on hot disinfection)
A configuration of the matrix by cast/foam additionally provides mechanical stability (less compliance/compressive volume stiffness in the system produces shorter cycle times in pressure hold tests)
A configuration of the matrix by cast/foam seals (if a hydraulic connection in the pipe system breaks)
The matrix effects vibration damping/an improvement of the acoustic properties of connected components
Foam as the matrix material provides the advantage of a smaller weight in comparison with a monoblock
Good accessibility of the connection points for the installation of the hydraulic unit at a dialysis machine is ensured, whereby an automated installation is made possible
Reduction of installation and material costs
Compact construction
Cost optimization of the additive production process by reduced material use (thin wall thicknesses and few down to no stiffening structures/elements necessary since the mechanical strength is achieved via the matrix or the foam/cast).

1. A medical device having a hydraulic unit that has a pipe system that is produced by means of an additive production process and whose intermediate spaces are at least partially filled by a matrix.

2. The medical device in accordance with claim 1, wherein that the pipe system is not self-supporting and is supported by the matrix.

3. The medical device in accordance with claim 1, wherein the pipe system is produced by means of 3D printing.

4. The medical device in accordance with claim 1, wherein the matrix comprises of a foam and/or a molding compound.

5. The medical device in accordance with claim 4, wherein the foam has closed pores and/or is a polyurethane foam.

6. The medical device in accordance with claim 1, wherein the pipe system is produced from a plastic satisfying medical demands and that the matrix is produced from another material that does not satisfy the medical demands.

7. The medical device in accordance with claim 1, wherein the pipe system is configured free of tubes.

8. The medical device in accordance with claim 1, wherein the pipe system is designed in one piece with a housing at least partially surrounding the pipe system, with the housing having at least one opening for introducing the matrix into the housing.

9. A method of producing a hydraulic unit of a medical device comprising the steps:
producing a pipe system by means of an additive production process; and
at least partially filling intermediate spaces of the pipe system by means of a matrix.

10. The method in accordance with claim 9, wherein the pipe system is not self-supporting and is supported by the matrix.

11. The method in accordance with claim 9, wherein the pipe system is produced by means of 3D printing.

12. The method in accordance with claim 9, wherein the pipe system is produced from a plastic satisfying medical demands and that the matrix is produced from another material that does not satisfy the medical demands.

13. The method in accordance with claim 9, wherein the pipe system is designed free of tubes.

14. The method in accordance with claim 9, wherein the pipe system is configured in one piece with a housing at least partially surrounding the pipe system, with the housing having at least one opening for introducing the matrix into the housing and with the matrix being introduced into the housing through the at least one opening to fill intermediate spaces of the pipe system.

15. The method in accordance with claim 9, wherein in the step of the at least partial filling of intermediate spaces of the pipe system by means of a matrix, the pipe system is temporarily arranged in a mold that is removed after a hardening of the matrix.

16. The medical device in accordance with claim 1, wherein the medical device is a dialysis machine.

17. The medical device in accordance with claim 1, wherein the pipe system is produced by means of 3D printing by means of a continuous liquid interface production, a laser sintering, or other additive process.

18. The medical device in accordance with claim 1, wherein the pipe system is produced from a plastic that is a cyanate ester, satisfying medical demands and is suitable for hot disinfection and that the matrix is produced from another material that does not satisfy the medical demands.

19. The method in accordance with claim 9, wherein the medical device is a dialysis machine.

20. The method in accordance with claim 9, wherein the pipe system is produced from a plastic that is a cyanate ester, satisfying medical demands and is suitable for hot disinfection and that the matrix is produced from another material that does not satisfy the medical demands.
